(19) United States
(12) Patent Application Publication
(10) Pub. No.: US 2019/0330341 A1
(43) Pub. Date: Oct. 31, 2019

(54) USE OF CD47 ANTIBODIES

(71) Applicants: Matthias TOTZECK, Gelsenkirchen (DE); Tienush RASSAF, Essen (DE)

(72) Inventors: Matthias TOTZECK, Gelsenkirchen (DE); Tienush RASSAF, Essen (DE)

(21) Appl. No.: 16/476,743

(22) PCT Filed: Jan. 9, 2018
(86) PCT No.: PCT/EP2018/050437

§ 371 Date: Jul. 9, 2019

(30) Foreign Application Priority Data
Jan. 10, 2017 (EP) 17150813.8

(51) Int. Cl. C07K 16/28; A61P 9/10

(52) U.S. Cl. CPC C07K 16/2803; A61K 2039/54; A61P 9/10

(57) ABSTRACT

The present invention relates to CD47 antibodies or fragments thereof binding specifically to the CD47 immunogen for use in preventing or treating tissue injury in states of ischemia or reperfusion in an object whereby the CD47 antibodies or fragments thereof are introduced intravascularly at the or near by the place of ischemia or reperfusion.

The present invention relates to CD47 antibodies or fragments thereof binding specifically to the CD47 immunogen for use in preventing or treating tissue injury in states of ischemia or reperfusion in an object whereby the CD47 antibodies or fragments thereof are introduced intravascularly at the or near by the place of ischemia or reperfusion.

### PRIOR ART

CD47, first detected on ovarian carcinoma and also known as integrin-associated protein (IAP) is widely expressed in many cell types including blood cells, neurons and cardiomyocytes. The levels of CD47 are significantly increased in many human cancers including solid tumors and hematopoetic diseases. Actually, high expression patterns have been associated with poor prognosis of the subjects. The correlation of CD47 levels and disease inspired to develop target therapies using humanized antibodies directed against tumor cells expressing increased CD47.

The CD47 molecule is a member of the immunoglobulin superfamily and its structure is unique insofar that it contains five transmembrane regions, a single Ig-like extracellular domain and a short intracellular tail.

However, regulation mechanism of the CD47 expression is still under investigation. Of note, CD47 expression has been related to states of hypoxia. In experimental renal ischemia or pulmonary artery hypertension CD47 levels were significantly elevated (Soto-Pantoja, D. R., et al., Expert Opinions on Therapeutic Targets, 2013, 17:89-103). However, the role of CD47 in the heart remains incompletely determined. Two major functions of CD47 are presently discussed. The first major function of CD47 is the functional receptor for thrombospondines (TSP). The TSP family consists of five multimeric, multidomain calcium-binding extracellular matrix proteins. Upon binding to CD47, TSP regulates several processes within the cardiomyocyte including calcium homoeostasis, gene expression programs, the regulation of matrix metalloproteins and NO signaling. Emerging evidence appoints to a significant role for TSP in cardiac disease and increased levels have been found in pressure-load hypertrophie, heart failure and myocardial infarction. It is well known that NO has beneficial effects in myocardial functions in a dose dependent manner.

The second major function of CD47 is the regulation of macrophages. Interaction of CD47 with signal regulatory protein (SIRPα) on macrophages conveys a signaling and a recognition of cells meaning that the cell signals to the macrophages that they are self and should not be phagocyzed. Cells with low CD47 surface levels as in aged erythrocytes or with antibody-blocked CD47 receptor are primed for removal by the phagocytic machinery while tumor cells with high CD47 pattern avoid clearance. SIRPα represents an inhibitory receptor as a counterpart of CD47 expressed on phagocytes. Upon binding of CD47 by SIRPα a phosphorylation cascade is triggered downregulating the activity of the macrophages. In acute myocardic infarction (AMI) the recruitment of adequate phagocytosis is required. That is, NO-dependent manipulation of macrophage inhibitory factor (MIF), a component involved in macrophage attraction in diseased organs, could contribute to infarct reduction. On the other hand, macrophage malfunction can be deleterious. Phagocytes including macrophages interacting with the damaged cardiomyocytes resulting in proteolysis, deadcell removal, angiogenesis and wound healing allowing effecting of remodeling. Actually a timely removal of apotopic cells in the border zones of the AAR (area at risk) thus preventing the dissemination of injury throughout the myocardium. Based on tumor studies, CD47 has emerged a major regulator of macrophage function.

In EP 2 569 013 B1 humanized and chimeric monodonal antibodies against CD47 are described being beneficial in the treatment of cancer like acute myeloid leukemia. Further, EP 2 477 648 A1 describes a synergistic anti-CD47 therapy for hematologic cancers.

In WO 2015/191861 A1 therapeutic CD47 antibodies have been described blocking the binding of SIRPα and TSP1 to cancer, thus, promoting phagocytosis of susceptible cancer cells. Therein, also the use of the therapeutic CD47 antibodies in other diseases including treatment of ischemia/reperfusion injury, autoimmune or inflammatory disease, as well as various types of cancer have been identified without demonstrating any effect on the same except for cancer.

Reperfusion injury also known as re-oxygenation injury is the tissue damage caused when blood supply returns to the tissue after a period of ischemia or lack of oxygen, namely anoxia or hyperoxia. That is, absence of oxygen and nutrients from blood during the ischemic period may damage said tissue, thus, restoration of circulation results in inflammation and oxidative damage through the induction of oxidative stress rather than restoration of malfunction. Various types of reperfusion injury exist including various mechanisms. The treatment of reperfusion injury includes various approaches including therapeutic hypothermia and treatment with various compounds including cyclosporine.

Ischemia is known as a restriction in the blood supply to tissues, thus, causing a shortage of oxygen as well as nutrients required for cellular metabolism. Ischemia typically results in damage or disfunction of the surrounding tissue. Ischemia may occur in various of the bodies including cardiac ischemia also known as myocardial infarction as wells as in brain (stroke) in the bowel or the limb.

Recently, HUI-BO Wang et al., cellular physiology and biochemistry, 2016, 40, 1163-1174, identifies RNAi mediated down-regulation of CD47 protects against ischemia/reperfusion-induced myocardial damage via activation of eNOS in a rat model. Therein, a specific CD47 RNAi is used to down-regulate CD47 whereby in an experimental set-up rats were treated with said CD47 RNAi and reduction of CD47 was confirmed. It was demonstrated therein, that the time frame between the injection of the CD47 RNAi and experimental myocardial infarction is critical as this is required for the down-regulation of CD47.

However, iRNA based therapies have various disadvantages, as iRNA treatments aim to reduce target proteins to specifically evaluate signal transduction pathways. The disadvantages of siRNA based therapies include the need for a comparable long pre-treatment with siRNA and the incomplete inhibition of the targeted pathway, e.g. as summarized in Monaghan, M. et al, trends pharmacol. sci, 2012, 33 (12), 635-645. Since in the scope of cardio protection during a myocardial infarction and particularly during myocardial ischemia and reperfusion injury an immediate and complete blocking of a deleterious pathway is required, iRNA is not a suitable means for treating and protecting against ischemia/reperfusion-induced myocardial damage.

Although various approaches for treating ischemia and reperfusion are described in the art, there is still ongoing need to treat ischemia and reperfusion, thus, reducing damaging of tissue. In particular, early treatment is desired.

### BRIEF DESCRIPTION OF THE PRESENT INVENTION

In a first aspect, the present invention relates to CD47 antibodies or fragments thereof binding specifically to the CD47 immunogen for use in preventing or treating tissue injury in states of ischemia and reperfusion in a subject whereby the CD47 antibodies or fragments thereof are introduced intravascularly, preferably at the or near by the place of ischemia or reperfusion, in particular, into the coronary artery.

It has been recognized that administering the CD47 antibodies or fragments thereof intravascularly, preferably at or near by the place of ischemia or reperfusion increase the efficacy of the inhibition of tissue damage or tissue injury, in particular with respect to an immediate response. Thus, the acute injury phase will be limited.

In a further aspect, the present invention relates to a method for preventing or treating tissue injury in states of ischemia and reperfusion including the step of administering or introducing intravascularly, preferably at the or near by the place of ischemia or reperfusion CD47 antibodies or fragments thereof biding specifically to the CD47 immunogene.

### BRIEF DESCRIPTION OF THE DRAWINGS

FIG. 1: Effect of a CD47 therapeutic regimen on blood cell counts. (A) Experimental scheme. Mice were treated by i.p. injection with CD47 antibody at the shown time intervals. Blood was taken 90 min after the last injection. (B) No differences were detectable for hemoglobin and white blood cells in CD47Ab treated animals vs. control littermates (n=5, p>0.05, unpaired student's t-test, graph shows mean ±s.d.).

FIG. 2: Anti-CD47 antibody administration reduces infarct sizes. (A) Following anti-CD47Ab treatment or control IgG, infarct per AAR was significantly reduced vs. CTRL animals, while AAR was comparable in both groups. (B) This was mirrored by significantly reduced troponin levels as measured with a high sensitive assay as marker for myocardial injury. (*p<0.05, unpaired student's t-test, n=7 vs. 6, data are mean ±s.d.)

FIG. 3: FIG. 3 shows in (A) the neutrophil count in heart tissues following anti-CD47 antibody (Ab) treatment vs. control treatment using an irrelevant IgG antibody of the same isotype and ischemia/reperfusion injury at day 1 (d1) and day 3 (d3). (B) shows macrophages at d3, d5 and d7 following reperfusion (n=5-6,*P<0.05) with significant increases at day 7.

### DETAILED DESCRIPTION OF THE PRESENT INVENTION

In the first aspect, the present invention relates to CD47 antibodies or fragments thereof binding specifically to the CD47 immunogen for use in preventing or treating tissue injury in states of ischemia and reperfusion in a subject whereby the CD47 antibodies or fragments thereof are introduced intravascularly, preferably at the or near by the place of ischemia or reperfusion.

The present inventors recognized that tissue damage following ischemia/reperfusion can be reduced or inhibited by targeting CD47 on protein level in the vicinity of the area of ischemia or reperfusion. By binding to the cell surface receptor CD47 enhancing CD47 SERPα signaling the tissue injury in states of ischema and reperfusion can be reduced. In particular, it is possible to prophylactically or therapeutically treat tissue injury as a result of various disorders, conditions or diseases. Importantly, the present inventors recognized that only when introducing the CD47 antibodies or fragments thereof binding specifically to the CD47 immunogen intravascularly, preferably at or near by the place of ischemia or reperfusion, reduction of tissue injury can be achieved. This is particularly true when administering the CD47 antibodies or fragments thereof in the coronary artery. It has been recognized by the inventors that the reduction of tissue injury relies on the activation of endogenous phagocytic machinery for the removal of apoptotic cells in the promotion of adequate wound healing.

As used herein, the term “treatment” or “treating” refer to both the therapeutic treatment and prophylactic or preventative measures unless otherwise identified. Those in need of treatment include those already with the disorder as well as those in which the disorder is to be prevented or reducing the tissue injury and inhibiting any tissue injury.

The term “antibody” is used in the process and specifically covers monoclonal antibodies (including full length monoclonal antibodies), polyclonal antibodies and multi-specific antibodies (e.g. bi-specific antibodies) and antibody fragments so long as they inhibit the desired biological activity.

As used in this invention, the term “epitope” means any antigenic epitope determining an antigen to which the epitope of an antibody binds. Epitope determines usually consists of chemical active surface, groupings of molecules such as amino acids or sugar side chains and usually have specific three-dimensional structural characteristics, as well as specific charged characteristics.

The term “antibody fragment” and all variants thereof as used herein are defined as a portion of an intact antibody comprising the antigen binding side or variable region of the intact antibody. For example, the fragment is free of that constant heavy chain domains of the region of the intact antibody. Examples of any antibody fragments are Fab, Fab′, Fab′-SH, F(ab′)₂ and FV fragments; diabodies as well as single chain antibody fragments or single chain polypeptides.

The term “monoclonal antibody” as used herein refers to an antibody obtained from a population of substantially homogeneous antibody, i.e. the individual antibodies comprising the population of identical except for possible naturally occurring limitations that may be present in minor amounts. Monoclonal antibodies are highly specific, being directed against a single antigenic site. Each monoclonal antibody is directed against a single determinant on the antigen. In addition to their specificity, the monoclonal antibodies are advantageous in that they can be synthetized by hybridoma culture without any contamination with immunoglobulins. The monoclonal antibodies herein include hybrid and recombinant antibodies produced by splicing of a variable domain of anti-CD47 antibody with a constant domain, e.g. humanized antibodies, or a light-chain with a heavy chain, or a chain from one species with a chain form another species, or fusion with heterologous proteins, regard as of species of origin or immunoglobulin class or subclass designation as well as antibody fragments, as long as they exhibit the desired biological inhibit.

“Reperfusion” refers to restoration of blood flow to tissue that is ischemic, due to decreased blood flow. Reperfusion is a procedure for treating infarction or other ischemia, by enabling viable ischemic tissue to recover, thus limiting further necrosis. However, as noted before, reperfusion can itself further damage the ischemic tissue, causing the reperfusion injury. Further, ischemic/reperfusion injury involves tissue injury that occurs after blood flow is restored.

“Ischemia” refers to a vascular phenomenon in which a decrease in the blood supply to a bodily organ, tissue or part is caused, for instance by constriction or obstruction of one or more blood vessels. Ischemia sometimes results from vasoconstriction or thrombosis or embolism. Ischemia can lead to direct ischemic injury, tissue damage due to cell that is caused by reduces oxygen supply. Ischemia can occur acutely, as during surgery or from trauma to tissue incurred in accidents, injuries and wall settings, or following harvest of organ intended for subsequent transportation. It can also occur sub-acutely, as found in arteriosclerotic peripheral vascular disease, where progressive constriction of blood vessels leads to inadequate blood flow to tissue and organs. The resulted injury after transit decrease or interruption of blood flow involves two components, the direct injury occurring to ischemic interval and the indirect or reperfusion injury that follows.

The phrase “humanized antibodies” refers to monoclonal antibodies and antigen binding fragments thereof, including the antibody compounds described herein, that have binding in functional properties according to the disclosure similar to those disclosed herein and that have framework in consent regions that are substantially human or fully human surrounding CDRs deriving from a non-human antibody. In this connection, the term “framework region” or “framework sequence” refers to any one of the framework regions 1 to 4. Humanized antibodies and antigen binding fragments encompassed by the present disclosure include molecules wherein any one or more of the fragment regions 1 to 4 is substantially or fully human, i.e. wherein any of the possible combinations of individuals substantially or fully framework regions 1 to 4 is present. Substantially human frameworks are those that have at least 80% sequence identity to a known human germline framework sequence, preferably that substantially human framework have at least 85%, at least 88%, at least 90%, at least 91%, at least 92%, at least 93%, at least 94%, at least 95%, at least 96%, at least 97%, at least 98% or at least 99% sequence identity to a framework sequence as described or to a known human germline framework sequence.

As used herein, the phrase “sequence identity” means a percentage of identical nucleotide or amino acid residues at corresponding positions in two or more sequences when the sequence are known to maximize sequence matching, i.e. taking into account gaps and insertions. The skilled person is well aware of suitable methods and programs allowing calculation thereof accordingly, the same are publically available.

The terms “specifically combines”, “binds specifically”, “specific binding” and the like as applied to the present antibody compounds refer to the ability of a specific binding agent, the antibody or fragments thereof, to bind to a target molecular species containing the epitope in preference to binding to other molecular species with which the specific binding agent and target molecular species are mixed. A specific binding agent is said specifically to recognize a target molecular species when it can bind specifically to that target containing the epitope.

The singular terms “a”, “an” and “the” include plural reference under this context clearly indicates otherwise.

The term “comprising” as used herein is open-ended and means that the subject matter must contain all the features specifically recited therein, but there is no bar or additional features that are not recited being present as well. The term “comprising” or “comprise” are used interchangeably herein with the term “contain”. A specific embodiment of comprising or containing is consisting of.

The phrase “introduced . . . at the or near by the place of ischemia or reperfusion” refers to an administration or introduction of the antibodies or fragments thereof into the place of ischemia or reperfusion or in the vicinity of said place, thus, the antibodies or fragments thereof administered come immediately into contact with the place of ischemia or reperfusion. Thus, any deterioration or degradation of the effective agents, namely, the antibodies or fragments thereof, is avoided. The present inventors recognized that immediate administration is beneficial to reduce any tissue injury in states of ischemia reperfusion accordingly. Of note, the introduction is intravascular. In an embodiment the intravascular introduction at the or near by the place of ischemia or reperfusion is an administration into the coronary artery.

In an embodiment of the present invention, the object is a human and the CD47 immunogen is the immunogen having the sequence of SEQ ID No. 2 encoded e.g. by a nucleic acid sequence of SEQ ID No. 1 and CD47 antibodies or fragments thereof are humanized antibodies or fragments thereof.

It is preferred that the antibodies or the fragments thereof binding specifically to the CD47 immunogen of the object are modified in a way that the antibodies are tolerable by the object to be treated.

As used herein, the terms “subject” and “object” are used interchangeably. That is, an object is a subject and a subject is an object.

In an embodiment the object is a mammal, in particular a human.

In a further aspect, the present invention relates to the use of the CD47 antibodies or fragments thereof preventing or treating tissue injury in states of ischemia or reperfusion whereby the introduction or administration of said CD47 antibodies or fragments thereof into the object is by catheter, by infusion and/or other intravenous administration, by intraventricular administrations or combinations thereof.

For example, the introduction is by catheter into the reperfused coronary artery. Alternatively, introduction or administration is by both introduction of a loading dose by catheter into the coronary artery followed by a continuous intravenous infusion for a predetermined time, e.g. for 1 hour, for 12 hours, for 24 hours, for 2 days or 3 days.

Alternatively, the administration is by intravenous injection of a bolus followed by continues infusion. In another embodiment, the introduction is by intraventricular injection of a bolus or by intraventricular injection followed by a continuous intravenous infusion.

For example, the administration is regionally at the or near by the place of ischemia or reperfusion within the first 30 minutes, like the first 15 minutes, after ischemia or reperfusion and/or is followed by a continuous infusion.

In a further embodiment, the present invention relates to the CD47 antibodies or fragments thereof for use in preventing or treating tissue injury in states of ischemia or reperfusion wherein the ischemia and reperfusion is a result of
i) Myocardial infarction,
ii) Cardiac arrest and resuscitation,
iii) Coronary bypass surgery under the use of an extracorporal life support systems,
iv) Treatment of chronic coronary total occlusion lesions,
v) Treatment of type B aortic dissection using catheter interventional techniques,
vi) Stroke and interventional stroke treatment,
vii) Cardiogenic shock,
viii) Heart transplantation,
ix) Replacement of aortic valves or
x) High risk coronary interventions under the use of circulatory support systems.

In a further embodiment, the present invention relates to the CD47 antibodies or fragments thereof for use in preventing or treating tissue injury in states of ischemia or reperfusion in an object wherein the treatment is a prophylactic treatment before heart transplantation or replacement of vessels, in particular, artery and veins including aorta. As demonstrated in the examples, a prophylactic administration of the CD47 antibodies or fragments thereof reduces significantly the ischemic area in case of ischemia and reperfusion. Thus, any adverse effects due to the heart transplantation or replacement of vessels, in particular, artery and veins including aorta is reduced.

The skilled person is well aware of suitable methods for the prophylactic treatment, in particular, the prophylactic treatment before heart transplantation or replacement of vessels. Typically, the administration is at the or nearby the place of transplantation or replacement of vessels and, in addition, the transplanted organ, like the heart, or the newly vessel, including artery and veins, are prophylactically treated with the CD47 antibodies or fragments thereof binding specifically to the CD47 immunogen.

In a further embodiment, the present invention relates to the CD47 antibodies or fragments thereof for use in preventing or treating tissue injury in states of ischemia or reperfusion in an object undergoing treatment, in particular, surgery, including extra-corporal support systems. That is, to avoid or diminish any adverse side effects during treatment, the prophylactic or therapeutic administration of the CD47 antibodies or fragments thereof according to the present invention are beneficial. As identified, it is necessary to administrate the same at the or nearby the place of possible ischemia or reperfusion.

The term “at the or nearby the place of ischemia or reperfusion” includes embodiments, in particular, when administrated prophylactically, where the possible place of ischemia or reperfusion is considered to take place. Said term include embodiments where the administration is within or nearby the AAR.

In a further embodiment, the CD47 antibodies or fragments thereof according to the present invention are for use in preventing or treating tissue injury in states of ischemia or reperfusion wherein the intravascular introduction thereof at the or nearby the place of ischemia or reperfusion is in concert with an infusion of said CD47 antibodies or fragments.

That is, beside the local or regional initial treatment, the continued treatment is systematically, i.e. by infusion.

While the first, initial, prophylactic or therapeutic treatment is a local administration of the antibodies or fragments thereof, the further treatment is e.g. by continuous administration either systematically or locally. The skilled person is well aware of the suitable methods accordingly.

Further, the present invention provides the use of the CD47 antibodies or fragments thereof wherein the administration by intravascular introduction at the or nearby the place of ischemia or reperfusion is simultaneously or sequentially with the infusion of the CD47 antibodies or fragments thereof.

Moreover, the CD47 antibodies or fragments thereof for use in preventing or treating tissue injury in states of ischemia and reperfusion in an object is characterized in that the CD47 antibodies or fragments thereof are administrated by catheter at the or nearby the place of ischemia or reperfusion, in particular, a damaged organ, in a concentration sufficient to locally inhibit CD47 signaling, thus, enabling removing damaged cells by a pharmaceutic machinery for improving reperfusion injury.

That is, the present inventors recognized that after ischemia, CD47 expression is increased, thus, the “don't eat me” signal of the cells identifies themselves as cells not ready for phagocytosis by macrophages or the phagocytes.

In addition, the ischemia and or reperfusion increased at CD47 expression are not highest in the area of the severest damages, but the high levels are present in the boarder zones of the tissue damage (of the AAR) whereby these boarder zones are particularly important for the effects of cardio protective measures/therapies, and infarct-sparing effects are believed to be localized in this area.

Hence, the method and use according to the present invention targets the most important region, namely, the boarder zones of the damaged tissue or AAR, thus, inhibiting any spreading or propagation with the damaged tissue accordingly. Further, a wound healing is improved due to the activation of the phagocytic machinery or removing apoptotic cells and, in addition, promoting the adequate wound healing.

Also the increasing survive of ischemic tissue by targeting CD47 has been described before, e.g. Isenberg, J. S. at al, circulation research, 2007; 100; 712-720, only the present inventors recognized that in a first step the CD47 antibodies or fragments thereof are introduced intravascularly at the or nearby the place of ischemia or reperfusion.

Hence, in an embodiment the CD47 antibodies or fragments thereof are for use in preventing or treating tissue injury in states of ischemia or reperfusion in an object wherein the administration or introduction is by a combination of a first administration of an initiate dosage in form of intraventricular injection of a bolus or intravenous injection of a bolus or by catheter to the coronary artery followed by continuous infusion.

Moreover, the CD47 antibodies or fragments thereof particularly useful according to the present invention are CD47 antibodies or fragments thereof as described e.g. in WO 2011/143624 A2, in WO 2011/034969 A1 as well as WO 2015/191861 A1.

That is, in an embodiment of the present invention, the CD47 antibodies or fragments thereof for use in preventing or treating tissue injury in states of ischemia or reperfusion in an object an isolated chimeric or humanized antibody that specifically bind to human CD47, wherein said antibody comprises a heavy chain having each of the CDR sequences set forth in SEQ ID Nos. 3, 4, 5, or each of the CDR sequences set forth in SEQ ID Nos. 6, 7, 8, or each of the CDR sequences set forth in SEQ ID Nos. 9, 10, 11 and a light chain having each of the CDR sequences set forth in SEQ ID No. 12, 13, 14, or each of the CDR sequences set forth in SEQ ID Nos.15, 16, 17, or each of the CDR sequences set forth in SEQ ID Nos.18, 19, 20.

The CD47 antibodies or fragments thereof for use in preventing or treating tissue injury in states of ischemia or reperfusion in an object according to the present invention wherein the CD47 antibodies or fragments thereof are a monoclonal antibody or antigen binding fragment thereof which comprises a light chain variable region (LCVR) and a heavy chain variable region (HCVR), wherein said LCVR and said HCVR comprise, respectively, amino acid sequences selected from among the following combinations of one of the LCVRs of SEQ ID Nos. 21 to 45 and one of the HCVRs of SEQ ID Nos. 47 to 71:

wherein each one of the LCVR SEQ ID Nos 21 to 45 further comprises a constant domain having the amino acid sequence shown in SEQ ID No 46, and

wherein each one of the HCVR SEQ ID Nos 47 to 71 comprises a constant domain selected from among SEQ ID Nos 72, 73, 74, 75, 76.

The CD47 antibodies or fragments thereof for use in preventing or treating tissue injury in states of ischemia or reperfusion in an object according to the present invention wherein the antibody or antigen binding fragment thereof comprises three light chain complementary determining regions (LCDRs 1-3) and three heavy chain complementary determining regions (HCDRs 1-3), wherein:

LCDR 1 comprises the amino acid sequence RSSQSLVHSNGNTYLH (SEQ ID NO: 77)

LCDR 2 comprises the amino acid sequence KVSYRFS (SEQ ID NO:78); and

LCDR 3 comprises the amino acid sequence SQNTHVPRT (SEQ ID NO:79) ;

HCDR1 comprises the amino acid sequence GYTFTNYYVF (SEQ ID NO:80);

HCDR 2 comprises the amino acid sequence DINPVNGDTNFNEKFKN (SEQ ID NO:81); and

HCDR 3 comprises the amino acid sequence GGYTMDY (SEQ ID NO:82).

As demonstrated herein, the targeting of the CD47 protein using the CD47 antibodies or fragments thereof represents a suitable tool for treating ischemia or reperfusion in a subject as an immediate therapeutical approach. Namely, specific targeting of cells expressing CD47 is possible. In contrast, the approach based on siRNA techniques requiring a long pre-treatment and resulting in incomplete inhibition of the pathway. In particular, since typically targeting of siRNA into the cells is cell-type unspecific, large amounts of siRNA is required.

In contrast, the treatment with an antibody rather than with siRNA initiates a protective immunomodulatory signaling. This protect the heart in the acute phase of ischemia/reperfusion injury and induces a potentially beneficial remodeling of the affected myocardium in the later phases (days after ischemia and induction of reperfusion). While neutrophils are leucocytes that infiltrate the myocardium within minutes after injury, macrophages are largely responsible for the remodeling of the myocardium at later phases. As demonstrated herein, the present inventors are able to demonstrate that the infarct size is reduced in hearts treated with anti-CD47 antibodies. In addition, as demonstrated a modulation of immune cells occurs, namely, the anti-CD47 treatment induced a significant increase in neutrophil invasion at day 1 after treatment while macrophages are significantly increased at day 7.

In addition, the present invention relates to a method for treating ischemia or reperfusion in an object in need thereof for preventing or treating the tissue injury in states of ischemia or reperfusion comprising the step of administrating the CD47 antibodies or fragments thereof as defined herein, in particular, administrating the same intravascularly at the or near by the place of ischemia or reperfusion. The CD47 antibodies or fragments thereof binding specifically to the CD47 immunogen are administrated in effective dosages, that is, dosages are effected in preventing or treating the tissue injury in states of ischemia or reperfusion accordingly.

The skilled person is well aware of suitable dosages and can easily determine the effective amount accordingly.

Typically, the antibodies or fragments thereof are administrated as a pharmaceutically composition. The pharmaceutically compositions can be prepared by known methods. Typically, the pharmaceutic composition comprises a pharmaceutically or veterinary acceptable, e.g. physiologically acceptable, carrier, diluent or excipient.

As noted, the administration is intravascularly, preferably at the or nearby the place of ischemia or reperfusion. Of course, a combination with other known therapeutic methods are encompassed herein including the use of other antibodies or other active ingredients for preventing or treating the tissue injury in states of ischemia or reperfusion in an object accordingly.

The present invention will be described further by way of examples without limiting the same.

### Example 1 Effect of CD47 Antibody Administration on Hemoglobin and White Blood Cells

Following a seven day administration of CD47 antibody by i.v. injection blood samples had been acquired to determine the blood cell count of all blood cells in conjunction with hemoglobin levels, which had been compared to pretreatment levels. Additionally, blood cell counts and hemoglobin levels will had been assessed in every trial conducted under the usage of CD47 receptor blocking therapies.

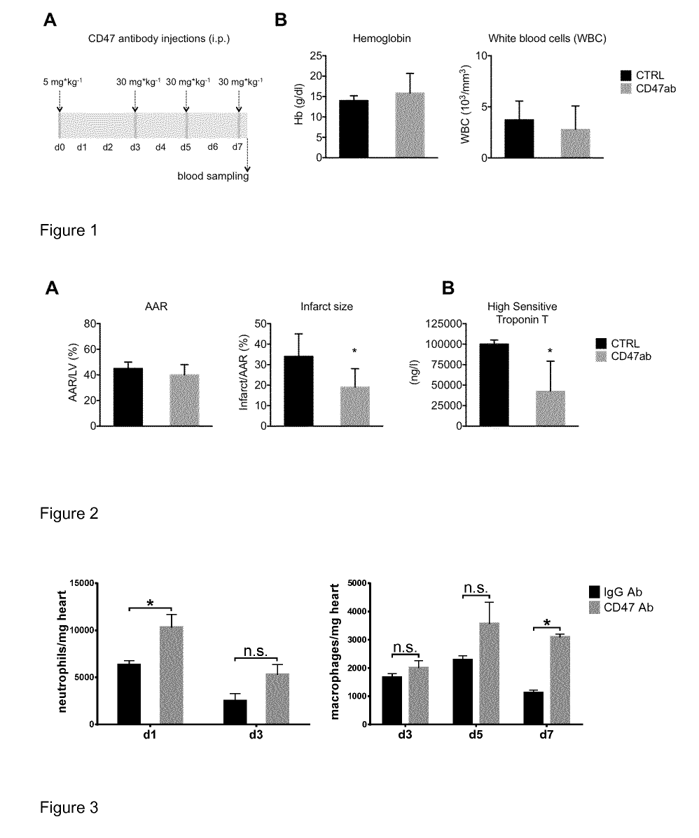

Previous studies in various preclinical cancer models have demonstrated that antibodies against CD47 can eliminate cancer cells or inhibit progression effectively. Herein, CD47 antibody administration had little to no side effects. Arguably, one of the highly discussed caveats of this approach is the interaction of the antibody with the recipient's red blood cells. Long-term blockade of CD47 on erythrocytes could enhance the clearance of these cells leading to anemia. Although this has not been the case in a relevant manner in the current preclinical tumor and transplantation studies, it was first set out to evaluate the effects on blood cells in mice after a seven day treatment with CD47 antibodies. FIG. 1A shows the therapeutic regimen schematically. C57BL/6 mice (twelve±three weeks old, male gender) were treated with an intraperitoneal (i.p.) injection of anti-CD47 antibody (CD47 ab, MIAP 301, Santa Cruz) on day 0 with a priming dose (5 mg*kg−1) and on days 3,5 and 7 (30 mg*kg−1) with a full therapeutic dose. This was compared against control injections at similar intervals. No injection was administered on days 1,2, 4 and 6. 90 min after the final dose, full blood was taken from the mouse heart, mixed with EDTA for anticoagulation and analyzed by flow cytometry. FIG. 1B shows the respective results after the 7d regimen for hemoglobin and white blood cells. The applicant could demonstrate that CD47Ab treatment did not affect these levels in comparison to CTRL littermates. This is a prerequisite for subsequent infarction studies.

As demonstrated, the CD47 treatment using the antibodies described did not affect the level of hemoglobin and white blood cells in control groups being a sign for usability of the same in the prevention or treatment of tissue injury in states of ischemia or reperfusion.

### Example 2

Based on the findings in Example 1, the efficacy of CD47 blockade was next tested in a mouse model of regional myocardial infarction. Mice received anti-CD47Ab according to the scheme outlined in FIG.1. 90 min after the last injection, anesthesia was initiated.15 A thoracotomy was performed in intubated and mechanically ventilated mice. I/R was induced by reversible ligation of the left coronary artery. Ischemia was maintained for a period of 30 min after which the suture was released. After surgical wound closure, mice recovered for 24 h. Hereafter, hearts were excised and treated according to previously published protocols (Rassaf et al. Circulation Res 2014 May 9; 114(10):1601-10. Doi: 10.1161/CIRCRESAHA.114303822).15 Infarct size was calculated against the AAR. Calculation was performed by two operators. FIG. 2A shows that anti-CD47Ab treatment reduced infarct size significantly with no significant difference on the size of the AAR as control. In parallel this caused a reduction in troponin levels as measured in the blood of the respective animals (FIG. 2B).

As noted before, hypoxia is believed to enhance CD47 expression. Surprisingly, also ischemia/reperfusion with a 24 h reoxygenation of the myocardium at risk induces CD47 expression in a relevant manner. The applicants therefore characterized CD47 levels in mouse hearts following in vivo I/R and 24 h of reperfusion. Western blotting was performed using mouse whole heart homogenates and anti-CD47 showing that CD47 was increased after I/R as compared to baseline control hearts. In contrast to what is expected, I/R increased CD47 expression not in the area of the severest hypoxia (inner myocardial borders), but at high levels at the border zones (data not shown). These border zones are particularly important for the effects of cardioprotective measures/therapies and infarct-sparing effects are believed to be localized at this area.

### Example 3 Determining Neutrophils in the Macrophages after CD47 Antibody Treatment and Deduction of Ischemia by Reversible Left Coronary Artery Ligation

Anti-CD47 antibody was administered to wild-type mice, and ischemia was induced by reversible left coronary artery litigation see Rassaf et al. Circ Res 2014, above. Hearts from treated animals were obtained following one day (d1) and three days (d3) for determination of neutrophils at d3, d5 and d7 for determining of macrophages. The results are shown in FIG. 3. After collagenase digestion, heart cells were counted by cytometry using labeling for neutrophils and macrophages and compared to control animals treated with control antibody, see Franck G et al Circ Res 2017 Jun. 23;121(1):31-42. doi: 10.1161/CIRCRESAHA.117.310694 and Hulsmans M et al. Cell. 2017 Apr. 20;169(3):510-522.e20. doi: 10.1016/j.ce11.2017.03.050. FIG. 3 shows that anti-CD47 treatment induces a significant increase in neutrophil invasion at day 1, while macrophages are significantly increased at day 7.

As demonstrated further, the efficacy of CD47 blocking agents harder can be determined in regional myocardial infarction. That is, as demonstrated with myocardial ischemia/reperfusion, a prophylactic treatment reduces significantly the infarct size compared to none retreated animals.

Taken together, the present inventors found that blocking of CD47 with a specific antibody therapy has no side effects on blood cell counts following the chosen application regimen. Furthermore antibody-dependent blockade of CD47 results in an acute decrease in myocardial I/R injury in an in vivo mouse regional model.

Further, siRNA is a technique for a chronic/permanent modification of a pathway, while an antibody-based approach inhibits target structures immediately and temporarily.

1-14. (canceled)

15. A method for preventing or treating tissue injury in states of ischemia and reperfusion in a subject comprising the step of administering CD47 antibodies or fragments thereof binding specifically to the CD47 immunogen, whereby the CD47 antibodies or fragments thereof are introduced intravascularly.

16. The method for preventing or treating tissue injury in states of ischemia and reperfusion in a subject according to claim 15 wherein administration is intravascularly at the place ischemia or reperfusion.

17. The method according to claim 16 wherein administration is into the coronary artery.

18. The method according to claim 15 Wherein the subject is a human and the CD47 immunogen is the immunogen having the sequence of SEQ ID No. 2 and the CD47 antibodies or fragments thereof are humanized antibodies or fragments thereof.

19. The method according to claim 15 wherein the administration by intravascular introduction is by catheter, by infusion and/or other intravenous administration, by intraventricular administration or combinations thereof.

20. The method according to claim 15 wherein the ischemia and reperfusion is a result of
i) myocardial infarction,
ii) cardiac arrest and resuscitation,
iii) coronary bypass surgery under the use of an extracorporal life support systems,
iv) treatment of chronic coronary total occlusion lesions,
v) treatment of type B aortic dissection using catheter interventional techniques,
vi) stroke and interventional stroke treatment,
vii) cardiogenic shock,
viii) heart transplantation,
ix) replacement of aortic valves or
x) high risk coronary interventions under the use of circulatory support systems.

21. The method according to claim 15 wherein administration is performed regionally at the or near by the place of ischemia or reperfusion within the first 30 minutes after ischemia or reperfusion.

22. The method according to claim 21 wherein administration is performed regionally within the first 15 minutes after ischemia or reperfusion.

23. The method according to claim 21 wherein administration is followed by continuous infusion of the CD47 antibodies or fragments thereof binding specifically to the CD47 immunogen.

24. The method according to claim 15 wherein administration is by continuous infusion.

25. The method according to claim 15 wherein the treatment is a prophylactic treatment before heart transplantation or replacement of vessels.

26. The method according to claim 25 wherein the replacement of vessels is replacement of artery and veins including aorta.

27. The method according to claim 15 wherein the subject is a subject undergoing treatment.

28. The method according to claim 27 wherein said subject is a subject undergoing surgery including extracorporal support systems.

29. The method according to claim 15 wherein the administration by intravascular introduction is in concert with an infusion of the said CD47 antibodies or fragments thereof.

30. The method according to claim 29 wherein the administration by intravascular introduction is at the place of ischemia or reperfusion.

31. The method according to claim 29 wherein the administration by intravascular introduction is simultaneously or sequentially with the infusion of said CD47 antibodies or fragments thereof.

32. The method according to claim 31 wherein the administration by intravascular introduction is at the place of ischemia or reperfusion.

33. The method according to claim 15 wherein the CD47 antibodies or fragments thereof are administered intravascularly by catheter for inhibiting locally CD47 signaling for removing damaged cells by phagocytic machinery for improving reperfusion injury.

34. The method according to claim 33 wherein the administration is at the place of ischemia or reperfusion.

35. The method according to claim 34 wherein the administration is by catheter in a damaged organ.

36. The method according to claim 15 wherein the administration is by a combination of a first administration of an initiate dosage in form of intraventricular injection of a bolus or intravenous injection of a bolus or by catheter to the coronary artery followed by continuous infusion.

37. The method according to claim 15 wherein the CD47 antibodies are an isolated chimeric or humanized antibody that specifically bind to human CD47, wherein said antibody comprises a heavy chain having each of the CDR sequences set forth in SEQ ID Nos. 3, 4, 5, or each of the CDR sequences set forth in SEQ ID Nos. 6, 7, 8, or each of the CDR sequences set forth in SEQ ID Nos. 9, 10, 11 and a light chain having each of the CDR sequences set forth in SEQ ID No. 12, 13, 14, or each of the CDR sequences set forth in SEQ ID Nos. 15, 16, 17, or each of the CDR sequences set forth in SEQ ID Nos. 18, 19, 20.

38. The method according to claim 15 wherein the CD47 antibodies or fragments thereof are a monoclonal antibody or antigen binding fragment thereof which comprises a light chain variable region (LCVR) and a heavy chain variable region (HCVR), wherein said LCVR and said HCVR comprise, respectively, amino acid sequences selected from among the following combinations of one of the LCVRs of SEQ ID Nos. 21 to 45 and one of the HCVRs of SEC, ID Nos. 47 to 71,
wherein each one of the LCVR SEQ ID Nos 21 to 45 further comprises a constant domain having the amino acid sequence shown in SEQ ID No 46, and
wherein each one of the HCVR SEQ ID Nos 47 to 71 comprises a constant domain selected from among SEQ ID Nos 72, 73, 74, 75, 76.

39. The method according to claim 38 wherein the antibody or antigen binding fragment thereof comprises three light chain complementary determining regions (LCDRs 1-3) and three heavy chain complementary determining regions (HCDRs 1-3), wherein:
LCDR 1 comprises the amino add sequence RSSQSLVHSNGNTYLH (SEQ ID NO:77)
LCDR 2 comprises the amino acid sequence KVSYRFS (SEQ ID NO:78);
LCDR 3 comprises the amino acid sequence SQNTHVPRT (SEQ ID NO:79);
HCDR 1 comprises the amino acid sequence GYTFTNYYVF (SEQ ID NO:80);
HCDR 2 comprises the amino acid sequence DINPVNGDTNFNEKFKN (SEQ ID NO:81): and
HCDR 3 comprises the amino acid sequence GGYTMDY (SEQ ID NO:82).


SEQUENCE LISTING (82 sequences)

SEQ ID NO: 1 - DNA, 879 bp, homo sapiens
  CDS (1)..(879)
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaa

SEQ ID NO: 2 - protein, 293 aa, homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 3 - protein, 5 aa, artificial
  Synthetic peptide
GYGMS

SEQ ID NO: 4 - protein, 17 aa, artificial
  Synthetic peptide
TITSGGTYTYYPDSVKG

SEQ ID NO: 5 - protein, 9 aa, artificial
  Synthetic peptide
SLAGNAMDY

SEQ ID NO: 6 - protein, 5 aa, artificial
  Synthetic peptide
NYNMH

SEQ ID NO: 7 - protein, 17 aa, artificial
  Synthetic peptide
TIYPGNDDTSYNQKFKD

SEQ ID NO: 8 - protein, 8 aa, artificial
  Synthetic peptide
GGYRAMDY

SEQ ID NO: 9 - protein, 5 aa, artificial
  Synthetic peptide
TYVVH

SEQ ID NO: 10 - protein, 17 aa, artificial
  Synthetic peptide
YINPYNDGTKYNEKFKG

SEQ ID NO: 11 - protein, 11 aa, artificial
  Synthetic peptide
GYYRYGYTMDY

SEQ ID NO: 12 - protein, 11 aa, artificial
  Synthetic peptide
RASQTISDYLH

SEQ ID NO: 13 - protein, 7 aa, artificial
  Synthetic peptide
FASQSIS

SEQ ID NO: 14 - protein, 9 aa, artificial
  Synthetic peptide
QNGHGFPRT

SEQ ID NO: 15 - protein, 16 aa, artificial
  Synthetic peptide
RSSQSIVYSNGNTYLG

SEQ ID NO: 16 - protein, 7 aa, artificial
  Synthetic peptide
KVSNRFS

SEQ ID NO: 17 - protein, 9 aa, artificial
  Synthetic peptide
FQGSHVPYT

SEQ ID NO: 18 - protein, 11 aa, artificial
  Synthetic peptide
RASQNFSDYLH

SEQ ID NO: 19 - protein, 7 aa, artificial
  Synthetic peptide
YVSHSIS

SEQ ID NO: 20 - protein, 9 aa, artificial
  Synthetic peptide
QNGHSFPPT

SEQ ID NO: 21 - protein, 106 aa, artificial
  Synthetic peptide
DVVMTQTPLSLSVSLGDQASISCRSSQSLVHSNGNTYLHWYLQKPGQSPKLLIYKVSYRFSGVPDRFSGSGSGTDFTLKISRVEAEDLGVYFCSQNTHVPRTFGQG

SEQ ID NO: 22 - protein, 106 aa, artificial
  Synthetic peptide
DIVMTQTPLSLPVTPGEPASISCRSSQSLVHSNGNTYLHWYQQKPGKAPKLLIYKVSYRFSGVPDRFSGSGSGTDFTLKISRVEAEDVGVYYCSQNTHVPRTFGQG

SEQ ID NO: 23 - protein, 106 aa, artificial
  Synthetic peptide
DVVMTQSPLSLPVTLGQPASISCRSSQSLVHSNGNTYLHWYQQKPGQAPRLLIYKVSYRFSGVPSRFSGSGSGTEFTLTISSLQPDDFATYYCSQNTHVPRTFGQG

SEQ ID NO: 24 - protein, 106 aa, artificial
  Synthetic peptide
DVVMTQSPLSLPVTLGQPASISCRSSQSLVHSNGNTYLHWYQQKPGKAPKLLIYKVSYRFSGVPDRFSGSGSGTDFTLKISRVEAEDVGVYYCSQNTHVPRTFGQG

SEQ ID NO: 25 - protein, 106 aa, artificial
  Synthetic peptide
DIQMTQSPSSLSASVGDRVTITCRSSQSLVHSNGNTYLHWYLQKPGQSPQLLIYKVSYRFSGIPARFSGSGSGTEFTLTISSLQSEDFAVYYCSQNTHVPRTFGQG

SEQ ID NO: 26 - protein, 106 aa, artificial
  Synthetic peptide
DIVMTQTPLSLPVTPGEPASISCRSSQSLVHSNGNTYLHWYLQKPGQSPQLLIYKVSYRFSGVPDRFSGSGSGTDFTLKISRVEAEDVGVYYCSQNTHVPRTFGQG

SEQ ID NO: 27 - protein, 106 aa, artificial; identical to SEQ ID NO: 25
  Synthetic peptide

SEQ ID NO: 28 - protein, 106 aa, artificial; identical to SEQ ID NO: 26
  Synthetic peptide

SEQ ID NO: 29 - protein, 106 aa, artificial; identical to SEQ ID NO: 22
  Synthetic peptide

SEQ ID NO: 30 - protein, 106 aa, artificial; identical to SEQ ID NO: 24
  Synthetic peptide

SEQ ID NO: 31 - protein, 106 aa, artificial; identical to SEQ ID NO: 26
  Synthetic peptide

SEQ ID NO: 32 - protein, 106 aa, artificial
  Synthetic peptide
EIVLTQSPATLSVSPGERATLSCRSSQSLVHSNGNTYLHWYQQKPGQAPRLLIYKVSYRFSGVPSRFSGSGSGTDFTFTISSLEAEDAATYYCSQNTHVPRTFGQG

SEQ ID NO: 33 - protein, 106 aa, artificial; identical to SEQ ID NO: 22
  Synthetic peptide

SEQ ID NO: 34 - protein, 106 aa, artificial; identical to SEQ ID NO: 22
  Synthetic peptide

SEQ ID NO: 35 - protein, 106 aa, artificial; identical to SEQ ID NO: 24
  Synthetic peptide

SEQ ID NO: 36 - protein, 106 aa, artificial
  Synthetic peptide
AIQLTQSPSSLSASVGDRVTITCRSSQSLVHSNGNTYLHWYQQKPGQAPRLLIYKVSYRFSGVPSRFSGSGSGTEFTLTISSLQPDDFATYYCSQNTHVPRTFGQG

SEQ ID NO: 37 - protein, 106 aa, artificial
  Synthetic peptide
AIQLTQSPSSLSASVGDRVTITCRSSQSLVHSNGNTYLHWYQQKPGQAPRLLIYKVSYRFSGVPDRFSGSGSGTDFTLKISRVEAEDVGVYYCSQNTHVPRTFGQG

SEQ ID NO: 38 - protein, 106 aa, artificial; identical to SEQ ID NO: 37
  Synthetic peptide

SEQ ID NO: 39 - protein, 106 aa, artificial; identical to SEQ ID NO: 32
  Synthetic peptide

SEQ ID NO: 40 - protein, 106 aa, artificial; identical to SEQ ID NO: 24
  Synthetic peptide

SEQ ID NO: 41 - protein, 106 aa, artificial; identical to SEQ ID NO: 26
  Synthetic peptide

SEQ ID NO: 42 - protein, 106 aa, artificial; identical to SEQ ID NO: 37
  Synthetic peptide

SEQ ID NO: 43 - protein, 106 aa, artificial; identical to SEQ ID NO: 32
  Synthetic peptide

SEQ ID NO: 44 - protein, 106 aa, artificial; identical to SEQ ID NO: 22
  Synthetic peptide

SEQ ID NO: 45 - protein, 106 aa, artificial; identical to SEQ ID NO: 36
  Synthetic peptide

SEQ ID NO: 46 - protein, 113 aa, artificial
  Synthetic peptide
TKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 109 aa, artificial
  Synthetic peptide
EVQLQQFGAELVKPGASMKLSCKASGYTFTNYYVFWVKQRPGQGLEWIGDINPVNGDTNFNEKFKNKATLTVDKSSTTTYLQLSSLTSEDSAVYYCTRGGYTMDYWGQG

SEQ ID NO: 48 - protein, 109 aa, artificial
  Synthetic peptide
QVQLQESGPGLVKPSQTLSLTCTVSGYTFTNYYVFWVRQARGQRLEWIGDINPVNGDTNFNEKFKNRVTISADKSISTAYLQWSSLKASDTAMYYCARGGYTMDYWGQG

SEQ ID NO: 49 - protein, 109 aa, artificial
  Synthetic peptide
QVQLQESGPGLVKPSQTLSLTCTVSGYTFTNYYVFWVRQARGQRLEWIGDINPVNGDTNFNEKFKNRVTISADKSISTAYLQWSSLKASDTAMYYCARGGYTMDYWGQA

SEQ ID NO: 50 - protein, 109 aa, artificial
  Synthetic peptide
EVQLVQSGAEVKKPGESLRISCKGSGYTFTNYYVFWVRQARGQRLEWIGDINPVNGDTNFNEKFKNRVTITADKSTSTAYMELSSLRSEDTAVYYCARGGYTMDYWGQG

SEQ ID NO: 51 - protein, 109 aa, artificial
  Synthetic peptide
QVQLQESGPGLVKPSQTLSLTCTVSGYTFTNYYVFWVRQAPGKGLEWVSDINPVNGDTNFNEKFKNRVTISVDTSKNQFSLKLSSVTAADTAVYYCARGGYTMDYWGQG

SEQ ID NO: 52 - protein, 109 aa, artificial; identical to SEQ ID NO: 51
  Synthetic peptide

SEQ ID NO: 53 - protein, 109 aa, artificial
  Synthetic peptide
QVQLQESGPGLVKPSQTLSLTCTVSGYTFTNYYVFWVRQARGQRLEWIGDINPVNGDTNFNEKFKNRLTISKDTSKNQVVLTMTNMDPVDTATYYCARGGYTMDYWGQG

SEQ ID NO: 54 - protein, 109 aa, artificial
  Synthetic peptide
QVQLQESGPGLVKPGATVKISCKVSGYTFTNYYVFWVRQARGQRLEWIGDINPVNGDTNFNEKFKNRVTITADKSTSTAYMELSSLRSEDTAVYYCARGGYTMDYWGQG

SEQ ID NO: 55 - protein, 109 aa, artificial
  Synthetic peptide
QITLKESGPTLVKPTQTLTLTCTFSGYTFTNYYVFWIRQSPSRGLEWLGDINPVNGDTNFNEKFKNRFTISRDNAKNSLYLQMNSLRAEDTAVYYCARGGYTMDYWGQG

SEQ ID NO: 56 - protein, 109 aa, artificial; identical to SEQ ID NO: 53
  Synthetic peptide

SEQ ID NO: 57 - protein, 109 aa, artificial; identical to SEQ ID NO: 53
  Synthetic peptide

SEQ ID NO: 58 - protein, 109 aa, artificial; identical to SEQ ID NO: 54
  Synthetic peptide

SEQ ID NO: 59 - protein, 109 aa, artificial; identical to SEQ ID NO: 54
  Synthetic peptide

SEQ ID NO: 60 - protein, 109 aa, artificial
  Synthetic peptide
EVQLVQSGAEVKKPGESLRISCKGSGYTFTNYYVFWIRQSPSRGLEWLGDINPVNGDTNFNEKFKNRVTITADKSTSTAYMELSSLRSEDTAVYYCARGGYTMDYWGQG

SEQ ID NO: 61 - protein, 109 aa, artificial; identical to SEQ ID NO: 48
  Synthetic peptide

SEQ ID NO: 62 - protein, 109 aa, artificial; identical to SEQ ID NO: 53
  Synthetic peptide

SEQ ID NO: 63 - protein, 109 aa, artificial; identical to SEQ ID NO: 48
  Synthetic peptide

SEQ ID NO: 64 - protein, 109 aa, artificial
  Synthetic peptide
EVQLVQSGAEVKKPGATVKISCKVSGYTFTNYYVFWIRQPPGKGLEWIGDINPVNGDTNFNEKFKNRVTITADKSTSTAYMELSSLRSEDTAVYYCARGGYTMDYWGQG

SEQ ID NO: 65 - protein, 109 aa, artificial; identical to SEQ ID NO: 60
  Synthetic peptide

SEQ ID NO: 66 - protein, 109 aa, artificial; identical to SEQ ID NO: 60
  Synthetic peptide

SEQ ID NO: 67 - protein, 109 aa, artificial
  Synthetic peptide
QITLKESGPTLVKPTQTLTLTCTFSGYTFTNYYVFWVRQAPGQGLEWMGDINPVNGDTNFNEKFKNRVTITADKSTSTAYMELSSLRSEDTAVYYCARGGYTMDYWGQG

SEQ ID NO: 68 - protein, 109 aa, artificial; identical to SEQ ID NO: 53
  Synthetic peptide

SEQ ID NO: 69 - protein, 109 aa, artificial; identical to SEQ ID NO: 53
  Synthetic peptide

SEQ ID NO: 70 - protein, 109 aa, artificial; identical to SEQ ID NO: 53
  Synthetic peptide

SEQ ID NO: 71 - protein, 109 aa, artificial; identical to SEQ ID NO: 54
  Synthetic peptide

SEQ ID NO: 72 - protein, 337 aa, artificial
  Synthetic peptide
TLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 245 aa, artificial
  Synthetic peptide
TLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSR

SEQ ID NO: 74 - protein, 337 aa, artificial
  Synthetic peptide
WGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYNTTPPMLDSDGSFFLYSKLTVDKSRWQQGNVFSCSVMHEALHNHYTQKSLSLSPGK

SEQ ID NO: 75 - protein, 334 aa, artificial
  Synthetic peptide
TTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 334 aa, artificial
  Synthetic peptide
TTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 16 aa, artificial
  Synthetic peptide
RSSQSLVHSNGNTYLH

SEQ ID NO: 78 - protein, 7 aa, artificial
  Synthetic peptide
KVSYRFS

SEQ ID NO: 79 - protein, 9 aa, artificial
  Synthetic peptide
SQNTHVPRT

SEQ ID NO: 80 - protein, 10 aa, artificial
  Synthetic peptide
GYTFTNYYVF

SEQ ID NO: 81 - protein, 16 aa, artificial
  Synthetic peptide
DINPVNGDTNFNEKFK

SEQ ID NO: 82 - protein, 7 aa, artificial
  Synthetic peptide
GGYTMDY